(19) United States
(12) Patent Application Publication
(10) Pub. No.: US 2024/0374723 A1
(43) Pub. Date: Nov. 14, 2024

(54) Induction of Antigen Specific Immunological Tolerance Using Inducible Pluripotent Stem Cell Derived Veto Cells

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Courtney Bartlett, Niceville, FL (US); Timothy Warbington, Phoenix, AZ (US)

(73) Assignee: CREATIVE MEDICAL TECHNOLOGIES, INC., Phoenix, AZ (US)

(21) Appl. No.: 18/646,563

(22) Filed: Apr. 25, 2024

Related U.S. Application Data
(60) Provisional application No. 63/465,616, filed on May 11, 2023.

(51) Int. Cl. A61K 39/00; C12N 5/0784

(52) U.S. Cl. CPC A61K 39/4615; A61K 39/4621; A61K 39/4622; C12N 5/0639; C12N 2501/22; C12N 2501/2304; C12N 2510/00

(57) ABSTRACT

The invention provides methods of inducing immunological tolerance to a transplanted cellular population or organ by creating and administering a “veto-like” cell originating from the identical genetic background of the organ donor. In one embodiment said artificially generated veto cell is a dendritic cell population possessing molecules associated with tolerance induced in natural anatomical locations such as the placenta, the testis, or the eye. In one embodiment dendritic cells resistant to maturation are generated from pluripotent stem cells that have been gene edited to lack genes needed for acquisition of antigen presenting properties such as relB, NF-kappa B and transporter associated protein. In another embodiment immature dendritic cells are gene edited/transfected to express tolerance associated molecules such as interleukin-10, interleukin-35, Fas ligand, TRAIL, TGF-beta, HLA-G and arginase.

### CROSS-REFERENCE TO RELATED APPLICATIONS

The present application claims benefit of Provisional Application Ser. No. 63/465,616, filed on May 11, 2023, entitled INDUCTION OF ANTIGEN SPECIFIC IMMUNOLOGICAL TOLERANCE USING INDUCIBLE PLURIPOTENT STEM CELL DERIVED VETO CELLS, the contents of which are incorporated herein by reference in its entirety.

### FIELD OF THE INVENTION

The invention relates to altered immune cells and their use in methods to alter the immune system in a mammal. More specifically, the invention is directed to the generation of donor specific “veto cells” capable of enhancing organ survival in the allogeneic and/or xenogeneic scenarios.

### BACKGROUND OF THE INVENTION

Immunological tolerance is vital in a healthy immune system to prevent excessive inflammation and autoimmunity, and many cell types contribute to tolerance. Immune tolerance is the functional unresponsiveness of the immune system towards cells or tissues. During healthy immune function, tolerance against self-tissues is maintained. However, the breakdown of tolerance can lead to autoimmune disease, conditions in which the immune system mistakenly recognizes and attacks host tissues and cells. Tissue destruction in autoimmune disease is driven by T and B cells specific for self-antigens which become aberrantly activated. These cells also secrete proteins—antibodies and inflammatory cytokines—that play major roles in the inflammatory effects occurring during autoimmunity. Autoimmune disease affects men and women of all ages, with some of the most prevalent autoimmune diseases including multiple sclerosis (MS), type 1 diabetes (T1D), rheumatoid arthritis and lupus. Transplantation is another area where similar inflammatory processes occur, but in a different context. In particular, a lack of tolerance of a recipient to a donor can result in the destruction of allogenic transplants—such as organs—because the tissue is seen as foreign by the immune cells of the recipient. Other areas where a lack of immune tolerance can negatively impact human health are allergic reactions and immune responses generated against recombinant protein drugs or viral vectors used for gene delivery. In allergies, for example, an immune response against otherwise harmless substances can trigger excess inflammation. This pathology is generally driven by allergen-specific B cells that generate IgE antibodies. In the case of immune responses against protein drugs or viral vectors, repeated treatment with these drugs can raise neutralizing antibodies in the patient that bind the proteins and inhibit their function upon subsequent administration. Thus, there are significant opportunities to improve new therapies involving immune tolerance across several important disease areas. It is believed that self-tolerance is largely mediated by FOXP3+ regulatory T cells (Treg) while type 1 regulatory T cells (Tr1) contribute greatly to peripheral tolerance and to some degree, self-tolerance. The ability to induce tolerance, termed “tolerogenesis” is considered the “Holy Grail” of Immunology. Unfortunately, progress towards clinical tolerogenesis has been slow at best.

Attempts at induction of tolerance have been made with Tr1 cells. These cells were first discovered when a severe combined immunodeficiency (SCID) patient received a mismatched allogeneic fetal liver and thymus transplant and developed stable mixed chimerism. This patient had high levels of serum IL-10, and further analysis revealed that T cell clones from this patient produced substantial amounts of IL-10 rapidly after T cell receptor (TCR) stimulation but produced low amounts of IL-2. Interestingly, IL-2 production was rescued by stimulating the cells in a TCR-independent manner. Follow-up studies established that Tr1 cells were a subset of regulatory T cells that produced large amounts of IL-10 and TGF-β, variable amounts of IFN-γ comparable to undifferentiated CD4+ T cells (Th0), and little to no IL-2 and IL-4, therefore making the cytokine profile distinct from Th1 and Th2 cells. Other approaches to inhibit unwanted destruction of healthy cells have focused on the administration of immunosuppressants which broadly suppress inflammatory function in immune cells. These include anti-inflammatory agents or corticosteroids. Recently more specific treatments have been applied, including recombinant cytokines and monoclonal antibodies. Antibody treatments function to deplete T or B cells, stop inflammatory function, or restrict migration of self-reactive immune cells. However, although antibody-based therapies are more specific, they are not selective in the sense that they bind their target receptor on all cells displaying these molecules. Thus, these therapies exert effects on both autoreactive immune cells and on immune cells exhibiting normal function. This lack of selectivity can leave patients immunocompromised or susceptible to specific types of infection. Further, existing treatments are not curative and must be repeatedly administered, often for life. These drawbacks highlight the potential impact of engineering more effective and safer therapies to promote immune tolerance.

### SUMMARY OF THE INVENTION

Preferred methods include embodiments of inducing antigen specific tolerance to a transplanted organ or collection of cells comprising: a) creating a pluripotent stem cell from the same individual as the source of the organ or cells to be transplanted; b) inducing differentiation of said cells into cells resembling cells possessing tolerogenic properties; c) administering said cells into a patient in need of an organ or cellular transplant.

Preferred methods include embodiments wherein said pluripotent stem cell is created from the donor using inducible pluripotent stem cell technology.

Preferred methods include embodiments wherein said pluripotent stem cell is created from the donor by extracting a donor egg and inducing the process of parthenogenesis.

Preferred methods include embodiments wherein parthenogenesis is induced using an adult tissue derived stem cell.

Preferred methods include embodiments wherein said parthenogenesis is stimulated by induction of calcium flux.

Preferred methods include embodiments wherein said parthenogenesis is performed using bone marrow stem cells.

Preferred methods include embodiments wherein said bone marrow stem cells are aldehyde dehydrogenase positive.

Preferred methods include embodiments wherein said bone marrow stem cells express one or more ABC transporter pumps.

Preferred methods include embodiments wherein said bone marrow stem cells are transferred cytoplasm of embryonic stem cells before being subject to parthenogenesis.

Preferred methods include embodiments wherein said bone marrow stem cells are transferred cytoplasm of inducible pluripotent stem cells before being subject to parthenogenesis.

Preferred methods include embodiments wherein said pluripotent stem cell is generated using somatic cell nuclear transfer technology.

Preferred methods include embodiments wherein said nucleus for creation of pluripotent stem cells is extracted from cells possessing the marker CD133.

Preferred methods include embodiments wherein said nucleus for creation of pluripotent stem cells is extracted from cells possessing the marker CD34.

Preferred methods include embodiments wherein said nucleus for creation of pluripotent stem cells is extracted from cells possessing the marker c-kit.

Preferred methods include embodiments wherein said nucleus for creation of pluripotent stem cells is extracted from cells possessing the marker CD123.

Preferred methods include embodiments wherein said nucleus for creation of pluripotent stem cells is extracted from cells possessing the marker CD127.

Preferred methods include embodiments wherein said nucleus for creation of pluripotent stem cells is extracted from cells possessing the marker CD174.

Preferred methods include embodiments wherein said nucleus for creation of pluripotent stem cells is extracted from cells that have been treated with a chromatin remodeling agent.

Preferred methods include embodiments wherein said chromatin remodeling agent is a histone deacetylase inhibitor.

Preferred methods include embodiments wherein said chromatin remodeling agent is a DNA methyltransferase inhibitor.

Preferred methods include embodiments wherein said chromatin remodeling agent is a GSK-3 inhibitor.

Preferred methods include embodiments wherein said chromatin remodeling agent is valproic acid.

Preferred methods include embodiments wherein said chromatin remodeling agent is trichostatin A.

Preferred methods include embodiments wherein said chromatin remodeling agent is phenylbutyrate.

Preferred methods include embodiments wherein said chromatin remodeling agent is sodium phenylbutyrate.

Preferred methods include embodiments wherein said chromatin remodeling agent is hydrogen gas.

Preferred methods include embodiments wherein said chromatin remodeling agent is hydrogen sulfide.

Preferred methods include embodiments wherein said chromatin remodeling agent is lithium oxide.

Preferred methods include embodiments wherein said cells resembling cells possessing tolerogenic properties are B cells.

Preferred methods include embodiments wherein said B cells express immunoglobulin Fc receptor I.

Preferred methods include embodiments wherein said B cells express immunoglobulin Fc receptor II.

Preferred methods include embodiments wherein said B cells express immunoglobulin Fc receptor III.

Preferred methods include embodiments wherein said B cells express CD19.

Preferred methods include embodiments wherein said B cells express CD20.

Preferred methods include embodiments wherein said B cells express PD-L1.

Preferred methods include embodiments wherein said B cells express autocrine IL-10.

Preferred methods include embodiments wherein said B cells express CD5.

Preferred methods include embodiments wherein said B cells express ICOS.

Preferred methods include embodiments wherein said B cells express HLA-G.

Preferred methods include embodiments wherein said B cells express soluble HLA-G.

Preferred methods include embodiments wherein said B cells produce TGF-beta.

Preferred methods include embodiments wherein said B cells are differentiated from said pluripotent stem cells by culture of said pluripotent stem cells in Whitte-Locke cultures.

Preferred methods include embodiments wherein said Whitte-Locke culture is established by culture of said pluripotent stem cells on a stromal layer of cells.

Preferred methods include embodiments wherein said stromal layer of cells produce an environment conducive for pluripotent stem cell differentiation into the lymphoid lineage.

Preferred methods include embodiments wherein said stromal layer of cells produce an environment conducive for pluripotent stem cell differentiation into the B cell lymphoid lineage.

Preferred methods include embodiments wherein said stromal layer of cells are bone marrow fibroblasts.

Preferred methods include embodiments wherein said stromal layer of cells are bone marrow mesenchymal stem cells.

Preferred methods include embodiments wherein said fibroblasts are pretreated with prostaglandin E2.

Preferred methods include embodiments wherein said bone marrow stem cells are pretreated with TNF-alpha at a concentration and time sufficient to stimulate said bone marrow stem cells to produce interleukin-7 at a concentration of at least 15 ng/million cells.

Preferred methods include embodiments wherein said bone marrow stem cells are pretreated with TNF-alpha at a concentration and time sufficient to stimulate said bone marrow stem cells to produce interleukin-3 at a concentration of at least 10 ng/million cells.

Preferred methods include embodiments wherein said bone marrow stem cells are pretreated with TNF-alpha at a concentration and time sufficient to stimulate said bone marrow stem cells to produce interleukin-15 at a concentration of at least 50 ng/million cells.

Preferred methods include embodiments wherein said bone marrow stem cells are pretreated with TNF-alpha at a concentration and time sufficient to stimulate said bone marrow stem cells to produce G-CSF at a concentration of at least 2 ng/million cells.

Preferred methods include embodiments wherein said bone marrow stem cells are pretreated with TNF-alpha at a concentration and time sufficient to stimulate said bone marrow stem cells to produce GM-CSF at a concentration of at least 2 ng/million cells.

Preferred methods include embodiments wherein said bone marrow stem cells are pretreated with TNF-alpha at a concentration and time sufficient to stimulate said bone marrow stem cells to produce G-CSF at a concentration of at least 7 ng/million cells.

Preferred methods include embodiments wherein said bone marrow stem cells are pretreated with TNF-alpha at a concentration and time sufficient to stimulate said bone marrow stem cells to produce M-CSF at a concentration of at least 2 ng/million cells.

Preferred methods include embodiments wherein said bone marrow stem cells are pretreated with TNF-alpha at a concentration and time sufficient to stimulate said bone marrow stem cells to produce osteopontin at a concentration of at least 2 ng/million cells.

Preferred methods include embodiments wherein said bone marrow stem cells are pretreated with TNF-alpha at a concentration and time sufficient to stimulate said bone marrow stem cells to produce leukemia inhibitor factor at a concentration of at least 10 ng/million cells.

Preferred methods include embodiments wherein said bone marrow stem cells are pretreated with interleukin-1 beta at a concentration and time sufficient to stimulate said bone marrow stem cells to produce interleukin-7 at a concentration of at least 15 ng/million cells.

Preferred methods include embodiments wherein said bone marrow stem cells are pretreated with interleukin-1 beta at a concentration and time sufficient to stimulate said bone marrow stem cells to produce interleukin-3 at a concentration of at least 10 ng/million cells.

Preferred methods include embodiments wherein said bone marrow stem cells are pretreated with interleukin-1 beta at a concentration and time sufficient to stimulate said bone marrow stem cells to produce interleukin-15 at a concentration of at least 50 ng/million cells.

Preferred methods include embodiments wherein said bone marrow stem cells are pretreated with interleukin-1 beta at a concentration and time sufficient to stimulate said bone marrow stem cells to produce G-CSF at a concentration of at least 2 ng/million cells.

Preferred methods include embodiments wherein said bone marrow stem cells are pretreated with interleukin-1 beta at a concentration and time sufficient to stimulate said bone marrow stem cells to produce GM-CSF at a concentration of at least 2 ng/million cells.

Preferred methods include embodiments wherein said bone marrow stem cells are pretreated with interleukin-1 beta at a concentration and time sufficient to stimulate said bone marrow stem cells to produce G-CSF at a concentration of at least 7 ng/million cells.

Preferred methods include embodiments wherein said bone marrow stem cells are pretreated with interleukin-1 beta at a concentration and time sufficient to stimulate said bone marrow stem cells to produce M-CSF at a concentration of at least 2 ng/million cells.

Preferred methods include embodiments wherein said bone marrow stem cells are pretreated with interleukin-1 beta at a concentration and time sufficient to stimulate said bone marrow stem cells to produce osteopontin at a concentration of at least 2 ng/million cells.

Preferred methods include embodiments wherein said bone marrow stem cells are pretreated with interleukin-1 beta at a concentration and time sufficient to stimulate said bone marrow stem cells to produce leukemia inhibitor factor at a concentration of at least 10 ng/million cells.

Preferred methods include embodiments wherein said stromal layer of cells are monocytes.

Preferred methods include embodiments wherein said monocytes are transfected with interleukin-7.

Preferred methods include embodiments wherein said monocytes are gene edited with interleukin-7.

Preferred methods include embodiments wherein said monocytes are isolated based on expression of CD14.

Preferred methods include embodiments wherein said monocytes are isolated based on expression of CD16.

Preferred methods include embodiments wherein said monocytes are isolated based on expression CD14 and CD73.

Preferred methods include embodiments wherein said monocytes are isolated based on expression of CD14 and TGF-beta receptor.

Preferred methods include embodiments wherein said monocytes are isolated based on expression of CD14 and c-kit.

Preferred methods include embodiments wherein said monocytes are isolated based on expression of CD14 and CD123.

Preferred methods include embodiments wherein said monocytes are isolated from bone marrow.

Preferred methods include embodiments wherein said monocytes are isolated from umbilical cord blood.

Preferred methods include embodiments wherein said monocytes are isolated from peripheral blood.

Preferred methods include embodiments wherein said monocytes are extracted by leukapheresis.

Preferred methods include embodiments wherein said monocytes are isolated by plastic adherence.

Preferred methods include embodiments wherein said monocytes are isolated from mobilized peripheral blood.

Preferred methods include embodiments wherein said mobilization of monocyte progenitors from peripheral blood is accomplished by treatment of the subject with hyperoxia.

Preferred methods include embodiments wherein said hyperoxia is induced by exposure to hyperbaric oxygen.

Preferred methods include embodiments wherein said hyperoxia is induced by exposure to ozone.

Preferred methods include embodiments wherein said ozone is administered in a combination of ozone and oxygen.

Preferred methods include embodiments wherein said ozone is administered in a combination of ozone and argon.

Preferred methods include embodiments wherein said ozone is administered in a combination of ozone and neon.

Preferred methods include embodiments wherein said ozone is administered in a combination of ozone and krypton.

Preferred methods include embodiments wherein said ozone is administered in a combination of ozone and xenon.

Preferred methods include embodiments wherein said ozone is administered in the form of autohemotherapy.

Preferred methods include embodiments wherein said ozone is administered in the form of ozone an a physiologically-acceptable liquid.

Preferred methods include embodiments wherein said physiologically-acceptable liquid is saline.

Preferred methods include embodiments wherein said physiologically-acceptable liquid is plasma.

Preferred methods include embodiments wherein said physiologically-acceptable liquid is serum.

Preferred methods include embodiments wherein said physiologically-acceptable liquid is saline and albumin

Preferred methods include embodiments wherein said physiologically-acceptable liquid is saline and plasma

Preferred methods include embodiments wherein said physiologically-acceptable liquid is saline and serum.

Preferred methods include embodiments wherein said physiologically-acceptable liquid is saline and platelet rich plasma.

Preferred methods include embodiments wherein said physiologically-acceptable liquid is saline and platelet rich fibrin.

Preferred methods include embodiments wherein said physiologically-acceptable liquid is Ringer's solution.

Preferred methods include embodiments wherein said ozone is delivered at a concentration sufficient to increase plasma levels of SDF-1 to above 7 ng/ml of blood.

Preferred methods include embodiments wherein said ozone is delivered at a concentration sufficient to increase plasma levels of G-CSF to above 10 ng/ml of blood.

Preferred methods include embodiments wherein said ozone is delivered at a concentration sufficient to increase plasma levels of GM-CSF to above 50 ng/ml of blood.

Preferred methods include embodiments wherein said ozone is delivered at a concentration sufficient to increase plasma levels of M-CSF to above 30 ng/ml of blood.

Preferred methods include embodiments wherein said ozone is delivered at a concentration sufficient to increase plasma levels of interleukin-10 to above 10 ng/ml of blood.

Preferred methods include embodiments wherein said stromal layer of cells are thymic medullary epithelial cells.

Preferred methods include embodiments wherein said thymic medullary epithelial cells are derived in vitro by differentiation of pluripotent stem cells.

Preferred methods include embodiments wherein said thymic medullary epithelial cells are extracted from thymi obtained after cardiac surgery.

Preferred methods include embodiments wherein said thymic medullary epithelial cells are extracted from thymi which would be normally discarded as medical waste.

Preferred methods include embodiments wherein said thymic medullary epithelial cells are extracted from organ donors.

Preferred methods include embodiments wherein said thymic medullary epithelial cells are cultured in a liquid media.

Preferred methods include embodiments wherein said liquid media is selected from a group of tissue culture media comprising of: a) DMEM; b) alpha MEM; c) RPMI media; d) Iscove's media; and e) AIM-V media.

Preferred methods include embodiments wherein said B cells are engineered to express tolerogenic molecules.

Preferred methods include embodiments wherein said B cells are derived from B1 cells that are expanded from peripheral blood of the donor.

Preferred methods include embodiments wherein said engineering of said tolerogenic molecules onto said B cells is performed at the level of pluripotent stem cell.

Preferred methods include embodiments wherein said engineering of said tolerogenic molecules onto said B cells is performed at the level of B cell progenitor cell.

Preferred methods include embodiments wherein said engineering of said tolerogenic molecules onto said B cells is performed at the level of differentiated B cell.

Preferred methods include embodiments wherein said tolerogenic molecule is a molecule capable of inducing death of activated T cells.

Preferred methods include embodiments wherein said tolerogenic molecule is a molecule capable of inducing death of activated B cells.

Preferred methods include embodiments wherein said tolerogenic molecule is a molecule capable of inducing death of activated NK cells.

Preferred methods include embodiments wherein said tolerogenic molecule is a molecule capable of inducing death of activated NKT cells.

Preferred methods include embodiments wherein said tolerogenic molecule is a molecule capable of inducing death of activated gamma delta T cells.

Preferred methods include embodiments wherein said activated T cells are activated by recognition of administered engineered B cells.

Preferred methods include embodiments wherein said engineered B cells are administered prior to an organ or cellular transplant.

Preferred methods include embodiments wherein said engineered B cells are administered concurrent with an organ or cellular transplant.

Preferred methods include embodiments wherein said engineered B cells are administered subsequent to said organ or cellular transplant.

Preferred methods include embodiments wherein interleukin-2 is administered concurrently with said engineered B cells in order to accelerate induction of immune cell apoptosis in said immune cells interacting with administered B cells.

Preferred methods include embodiments wherein said tolerogenic molecule is fas ligand.

Preferred methods include embodiments wherein said tolerogenic molecule is lymphotoxin.

Preferred methods include embodiments wherein said tolerogenic molecule is necrosin.

Preferred methods include embodiments wherein said tolerogenic molecule is TNF-gamma.

Preferred methods include embodiments wherein said tolerogenic molecule is CD252.

Preferred methods include embodiments wherein said tolerogenic molecule is CD154.

Preferred methods include embodiments wherein said tolerogenic molecule is CD70.

Preferred methods include embodiments wherein said tolerogenic molecule is CD153.

Preferred methods include embodiments wherein said tolerogenic molecule is 4.1-BB ligand.

Preferred methods include embodiments wherein said tolerogenic molecule is CD253.

Preferred methods include embodiments wherein said tolerogenic molecule is TRANCE.

Preferred methods include embodiments wherein said tolerogenic molecule is Death Receptor 3 ligand.

Preferred methods include embodiments wherein said tolerogenic molecule is TALL-2.

Preferred methods include embodiments wherein said tolerogenic molecule is BLyS.

Preferred methods include embodiments wherein said tolerogenic molecule is HVEML.

Preferred methods include embodiments wherein said tolerogenic molecule is TNSF15.

Preferred methods include embodiments wherein said tolerogenic molecule is GITRL.

Preferred methods include embodiments wherein said tolerogenic molecule is ED1-A1.

Preferred methods include embodiments wherein said tolerogenic molecule is HLA-G.

Preferred methods include embodiments wherein said tolerogenic molecule is IL-10.

Preferred methods include embodiments wherein said tolerogenic molecule is IL-4.

Preferred methods include embodiments wherein said tolerogenic molecule is IL-13.

Preferred methods include embodiments wherein said tolerogenic molecule is IL-20.

Preferred methods include embodiments wherein said tolerogenic molecule is IL-35.

Preferred methods include embodiments wherein said tolerogenic molecule is indolamine 2,3 dioxygenase.

Preferred methods include embodiments wherein said tolerogenic molecule is arginase.

Preferred methods include embodiments wherein said tolerogenic molecule is ILT-3.

Preferred methods include embodiments wherein said tolerogenic molecule is ILT-4.

Preferred methods include embodiments wherein tolerogenic dendritic cells are generated from pluripotent stem cells by treatment of pluripotent stem cells with interleukin-4 and GM-CSF.

Preferred methods include embodiments wherein said dendritic cells are generated in a manner in which they are resistant to maturation stimuli.

Preferred methods include embodiments wherein resistance to maturation is created by gene editing to remove NF-kappa B.

Preferred methods include embodiments wherein resistance to maturation is created by gene editing to constitutively express i-kappa B.

Preferred methods include embodiments wherein resistance to maturation is created by gene editing to remove rel-B.

Preferred methods include embodiments wherein resistance to maturation is created by gene editing to remove CD40.

Preferred methods include embodiments wherein resistance to maturation is created by gene editing to remove CD80.

Preferred methods include embodiments wherein resistance to maturation is created by gene editing to remove CD86.

Preferred methods include embodiments wherein resistance to maturation is created by gene editing to remove interleukin-1.

Preferred methods include embodiments wherein resistance to maturation is created by gene editing to remove interleukin-2.

Preferred methods include embodiments wherein resistance to maturation is created by gene editing to remove interleukin-7.

Preferred methods include embodiments wherein resistance to maturation is created by gene editing to remove interleukin-12.

Preferred methods include embodiments wherein resistance to maturation is created by gene editing to remove interleukin-15.

Preferred methods include embodiments wherein resistance to maturation is created by gene editing to remove interleukin-18.

Preferred methods include embodiments wherein resistance to maturation is created by gene editing to remove interleukin-21.

Preferred methods include embodiments wherein resistance to maturation is created by gene editing to remove interleukin-23.

Preferred methods include embodiments wherein resistance to maturation is created by gene editing to remove interleukin-27.

Preferred methods include embodiments wherein resistance to maturation is created by gene editing to remove interleukin-17.

Preferred methods include embodiments wherein said dendritic cells are capable of cross presentation to CD8 T cells.

Preferred methods include embodiments wherein said CD8 T cells are cytotoxic.

Preferred methods include embodiments wherein said CD8 T cells are capable of producing interferon gamma upon CD3 crosslinking.

Preferred methods include embodiments wherein said CD8 T cells are capable of producing perforin upon CD3 crosslinking.

Preferred methods include embodiments wherein said CD8 T cells are capable of producing granzyme B upon CD3 crosslinking.

Preferred methods include embodiments wherein said CD8 T cells are capable of producing complement C3 upon CD3 stimulation.

Preferred methods include embodiments wherein said pluripotent stem cells are inducible pluripotent stem cells generated with increased efficacy as compared to the standard Yamamoto protocol.

Preferred methods include embodiments wherein the following steps are performed to generated “enhanced” inducible pluripotent stem cells (iPSC); a) the transferring a transcription factor and Jhdm1b into mammalian adult cells which are then cultured in an inducing medium to induce pluripotent stem cell clones, wherein the transcription factor is Oct4 alone, or a combination of Oct4, Klf4, and Sox2, or a combination of Oct4, Klf4, c-Myc, and Sox2; and b) culturing and expanding the induced pluripotent stem cell clones in a stem cell culture medium.

Preferred methods include embodiments wherein said protocol comprises the steps of: a) transferring a transcription factor and Jhdm1b into mammalian adult cells which are then cultured in an inducing medium containing vitamin C to induce pluripotent stem cell clones, wherein the transcription factor is Oct4 alone, or a combination of Oct4 and Sox2, or a combination of Oct4 and Klf4, or a combination of Oct4, Klf4, and Sox2; and b) culturing and expanding the induced pluripotent stem cell clones in a stem cell culture medium.

Preferred methods include embodiments comprising the following steps: a) transferring a transcription factor and Jhdm1b into mammalian adult cells which are then cultured in an inducing medium containing vitamin C to induce pluripotent stem cell clones, wherein the transcription factor is Oct4 alone, or a combination of Oct4 and Sox2, or a combination of Oct4 and Klf4, or a combination of Oct4, Klf4, and Sox2; and b). culturing and expanding the induced pluripotent stem cell clones in a stem cell culture medium containing vitamin C.

Preferred methods include embodiments wherein augmentation of efficacy of iPSC generation is performed by inducing expression of that the transcription factor and Jhdm1b are encoded or noncoding RNAs, proteins, or polypeptides capable of inducing pluripotent stem cells.

The method for increasing the efficiency of inducing pluripotent stem cells of any one of claim 184 or 185, characterized in that the transferring of Jhdm1b into mammalian adult cells is achieved by incorporating a vector capable of expressing Jhdm1b into the cells.

The method for increasing the efficiency of inducing pluripotent stem cells of claim 186, characterized in that the vector is a viral vector, a plasmid vector, an episomal vector, or an mRNA vector, or is chemically synthesized directly.

Preferred methods include embodiments wherein said viral vector is a retrovirus which is a pMXs vector.

A method of inducing tolerance to a transplanted organ or cell population comprising the steps of: a) extracting a cellular population from donor said organ or cell population; b) inducing dedifferentiation of said cellular population; c) generating a tolerogenic cell population by differentiating said dedifferentiated cell population in the presenting of gene adding factors, and/or gene subtracting factors; d) stimulating production of exosomes from said tolerogenic cells; and e) introducing said exosomes into a person in which tolerance induction to a specific organ or cellular population is desired.

Preferred methods include embodiments wherein said dedifferentiation of a cellular population is performed on cells possessing immature properties.

Preferred methods include embodiments wherein said cells possessing immature properties and/or dedifferentiated/reprogrammed properties are mesenchymal stem cells.

Preferred methods include embodiments wherein said mesenchymal stem cells are isolated from the bone marrow.

Preferred methods include embodiments wherein said mesenchymal stem cells are isolated from peripheral blood.

Preferred methods include embodiments wherein said mesenchymal stem cells are isolated from mobilized bone marrow.

Preferred methods include embodiments wherein said mesenchymal stem cells are isolated from skin.

Preferred methods include embodiments wherein said mesenchymal stem cells are isolated from the endometrium.

Preferred methods include embodiments wherein said mesenchymal stem cells are isolated from the fallopian tubes.

Preferred methods include embodiments wherein said mesenchymal stem cells are isolated from the omentum.

Preferred methods include embodiments wherein said mesenchymal stem cells are isolated from the cerebral spinal fluid.

Preferred methods include embodiments wherein said mesenchymal stem cells are isolated from adipose tissue.

Preferred methods include embodiments wherein said mesenchymal stem cells are isolated from deciduous teeth.

Preferred methods include embodiments wherein said mesenchymal stem cells are isolated from the hair follicle.

Preferred methods include embodiments wherein said mesenchymal stem cells are isolated from umbilical cord blood.

Preferred methods include embodiments wherein said mesenchymal stem cells are isolated from Wharton's jelly.

Preferred methods include embodiments wherein said cells are isolated by: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45, optionally one or more steps are performed in the presence of hypoxia, wherein hypoxia is sufficient to induce translocation of HIF-1 alpha.

Preferred methods include embodiments wherein dissociation of fetal vascular lobes is accomplishing by incubation with a mixture of about 2% collagenase, about 0.25% trypsin and about 0.1% DNAse in tissue culture medium.

Preferred methods include embodiments wherein dissociation of fetal vascular lobes is accomplishing by incubation with a mixture of about 2% collagenase, about 0.25% trypsin and about 0.1% DNAse in tissue culture medium.

Preferred methods include embodiments wherein cells isolated are comprised of adherent cells expressing the marker CD73 but substantially lacking CD105.

Preferred methods include embodiments wherein cells isolated are comprised of adherent cells expressing the marker CD73 and CD105 but lacking in CD90.

Preferred methods include embodiments wherein cells isolated are comprised of adherent cells expressing the marker CD73 and CD105 but lacking in CD56.

Preferred methods include embodiments wherein cells isolated are comprised of adherent cells expressing the marker CD73 and CD105 but lacking in CD123.

Preferred methods include embodiments wherein said reprogramming is induced by culture of said cells in a liquid media containing ascorbic acid.

Preferred methods include embodiments wherein said reprogramming is induced by culture of said cells in a liquid media containing transferrin.

Preferred methods include embodiments wherein said reprogramming is induced by culture of said cells in a liquid media containing sodium bicarbonate.

Preferred methods include embodiments wherein said reprogramming is induced by culture of said cells in a liquid media containing insulin.

Preferred methods include embodiments wherein said reprogramming is induced by culture of said cells in a liquid media containing sodium selenite.

Preferred methods include embodiments wherein said reprogramming is induced by culture of said cells in a hypoxic environment.

Preferred methods include embodiments wherein said hypoxic environment comprises of oxygen levels low enough to induce activation of hypoxia inducible factor (HIF)-1.

Preferred methods include embodiments wherein said hypoxic environment is culture of said cells in an environment less than 21% oxygen.

Preferred methods include embodiments wherein said hypoxic environment is culture of said cells in an environment less than 15% oxygen.

Preferred methods include embodiments wherein said hypoxic environment is culture of said cells in an environment less than 10% oxygen.

Preferred methods include embodiments wherein said hypoxic environment is culture of said cells in an environment containing approximately 5% oxygen.

Preferred methods include embodiments wherein said reprogramming is induced by culture of cells in a liquid media containing a MAP Kinase inhibitor.

Preferred methods include embodiments wherein said MAP kinase inhibitor is PD0325901/

Preferred methods include embodiments wherein said reprogramming is induced by culture of cells in a liquid media containing SB431542.

Preferred methods include embodiments wherein said reprogramming is induced by culture of cells in a liquid media containing CHIR99021.

Preferred methods include embodiments wherein said reprogramming is induced by culture of cells in a liquid media containing Y-27632.

Preferred methods include embodiments wherein said reprogramming is induced by culture of cells in a liquid media containing Y-thiazovivin.

Preferred methods include embodiments wherein said reprogramming is induced by culture of cells in a liquid media containing FGF-1.

Preferred methods include embodiments wherein said reprogramming is induced by culture of cells in a liquid media containing FGF-2.

Preferred methods include embodiments wherein said reprogramming is induced by culture of cells in a liquid media containing FGF-5.

Preferred methods include embodiments wherein said reprogramming is induced by culture of cells in a liquid media containing sodium borate.

Preferred methods include embodiments wherein said reprogramming is induced by culture of cells in a liquid media containing erythropoietin (EPO).

Preferred methods include embodiments wherein said reprogramming is induced by culture of cells in a liquid media containing interleukin-3.

Preferred methods include embodiments wherein said reprogramming is induced by culture of cells in a liquid media containing interleukin-6.

Preferred methods include embodiments wherein said reprogramming is induced by culture of cells in a liquid media containing interleukin-8.

Preferred methods include embodiments wherein said reprogramming is induced by culture of cells in a liquid media containing interleukin-10.

Preferred methods include embodiments wherein said reprogramming is induced by culture of cells in a liquid media containing interleukin-18.

Preferred methods include embodiments wherein said reprogramming is induced by culture of cells in a liquid media containing interleukin-20.

Preferred methods include embodiments wherein said reprogramming is induced by culture of cells in a liquid media containing interleukin-25.

Preferred methods include embodiments wherein said reprogramming is induced by culture of cells in a liquid media containing insulin-like growth factor-1 (IGF-1).

Preferred methods include embodiments wherein said reprogramming is induced by culture of cells in a liquid media containing dexamethasone.

Preferred methods include embodiments wherein said reprogramming is induced by culture of cells in a liquid media containing holo-transferrin.

Preferred methods include embodiments wherein said reprogramming is induced by culture of cells in a liquid media containing amino acids selected from a group comprising of Glycine, L-histidine, L-isoleucine, L-methionine, L-phenylalanine, L-proline, L-hydroxyproline, L-serine, L-threonine, L-tryptophan, and L-tyrosine, L-valine.

Preferred methods include embodiments wherein said reprogramming is induced by culture of cells in a liquid media containing vitamins and/or antioxidants selected from a group comprising of thiamine, reduced glutathione, ascorbic acid and 2-PO.sub.4.

Preferred methods include embodiments wherein said reprogramming is induced by culture of cells in a liquid media containing trace elements selected from a group comprising of: Ag.sup.+, Al.sup.3+, Ba.sup.2+, Cd.sup.2+, Co.sup.2+, Cr.sup.3+, Ge.sup.4+, Se.sup.4+, Br.sup.−, I.sup.−, F.sup.−, Mn.sup.2+, Si.sup.4+, V.sup.5+, MO.sup.6+, Ni.sup.2+, Rb.sup.+, Sn.sup.2+, and Zr.sup.4+.

Preferred methods include embodiments wherein said reprogramming is induced by culture of cells in a liquid media containing cytoplasm of an undifferentiated cell.

Preferred methods include embodiments wherein said cell being reprogrammed has its membrane temporarily permeabilized.

The method of 248, wherein said temporary permeabilization allows for entry of cytoplasm of undifferentiated cell into cytoplasm of said cell to be reprogrammed.

Preferred methods include embodiments wherein said undifferentiated cell is syngeneic with the cell whose reprogramming is desired.

Preferred methods include embodiments wherein said undifferentiated cell is allogeneic with the cell whose reprogramming is desired.

Preferred methods include embodiments wherein said undifferentiated cell is xenogeneic with the cell whose reprogramming is desired.

Preferred methods include embodiments wherein said permeabilization is mediated by electroporation.

Preferred methods include embodiments wherein said permeabilization is mediated by Streptolysin O treatment.

Preferred methods include embodiments wherein said permeabilization is mediated by transient treatment with complement membrane attack complex.

Preferred methods include embodiments wherein said permeabilization is mediated by transient treatment with perforin.

Preferred methods include embodiments wherein said permeabilization is mediated by transient treatment with granzyme.

Preferred methods include embodiments wherein said undifferentiated cell is an oocyte.

Preferred methods include embodiments wherein said oocyte is programmed to be at G0/G1 of cell cycle.

Preferred methods include embodiments wherein said programming to be at G0/G1 of cell cycle is accomplished by exposure to mitomycin C.

Preferred methods include embodiments wherein said programming to be at G0/G1 of cell cycle is accomplished by exposure to serum starvation.

Preferred methods include embodiments wherein said undifferentiated cell is an inducible pluripotent stem cell.

Preferred methods include embodiments wherein said undifferentiated cell is a parthenogenic derived stem cell.

Preferred methods include embodiments wherein said undifferentiated cell is an embryonic stem cell.

Preferred methods include embodiments wherein said undifferentiated cell is a somatic cell nuclear transfer derived stem cell.

Preferred methods include embodiments wherein said undifferentiated cell is a cytoplasmically reprogrammed stem cell.

Preferred methods include embodiments wherein said undifferentiated cell is a cell obtained by fusion of an adult cell with a pluripotent stem cell.

Preferred methods include embodiments wherein said fusion is accomplished by the use of polyethylene glycol.

Preferred methods include embodiments wherein said fusion is accomplished by the use of electrically mediated fusion.

Preferred methods include embodiments wherein said tolerogenic cell population is a mesenchymal stem cell.

Preferred methods include embodiments wherein said tolerogenic cell population is a B regulatory cell.

Preferred methods include embodiments wherein said tolerogenic cell population is a T regulatory cell.

Preferred methods include embodiments wherein said tolerogenic cell population is a monocytic lineage cell.

Preferred methods include embodiments wherein said tolerogenic cell population is a myeloid suppressor cell.

Preferred methods include embodiments wherein said tolerogenic cell population is a trophoblast cell.

Preferred methods include embodiments wherein said tolerogenic cell population is an extravillous trophoblast cell.

Preferred methods include embodiments wherein said tolerogenic cell population is an immature dendritic cell.

Preferred methods include embodiments wherein said tolerogenic cell population is a lymphoid dendritic cell.

Preferred methods include embodiments wherein said cells are induced to express HLA-G.

Preferred methods include embodiments wherein said cells are induced to express HLA-E.

Preferred methods include embodiments wherein said cells are induced to express ILT-3.

Preferred methods include embodiments wherein said cells are induced to express ILT-4.

Preferred methods include embodiments wherein said cells are induced to express TGF-alpha.

Preferred methods include embodiments wherein said cells are induced to express TGF-beta.

Preferred methods include embodiments wherein said cells are induced to express arginase.

Preferred methods include embodiments wherein said cells are induced to express indolamine 2,3 dioxygenase.

Preferred methods include embodiments wherein said cells are induced to express Fas ligand.

Preferred methods include embodiments wherein said cells are induced to express TRANCE.

Preferred methods include embodiments wherein said cells are induced to express iNOS.

Preferred methods include embodiments wherein said cells are induced to express glutaminase.

Preferred methods include embodiments wherein said cells are induced to express soluble TNF-alpha receptor p55.

Preferred methods include embodiments wherein said cells are induced to express soluble TNF-alpha receptor p75.

Preferred methods include embodiments wherein said cells are stimulated to produce exosomes by treatment with an inflammatory stimuli.

Preferred methods include embodiments wherein said inflammatory stimuli is an activator of the Janus activated kinase pathway.

Preferred methods include embodiments wherein said inflammatory stimuli is an activator of the signal transduction and activator of transcription pathway.

Preferred methods include embodiments wherein said exosomes are concentrated by affinity chromatography.

Preferred methods include embodiments wherein said exosomes are concentrated by size exclusion chromatography.

Preferred methods include embodiments wherein said exosomes are concentrated by affinity HPLC.

Preferred methods include embodiments wherein said exosomes are concentrated by affinity FPLC.

Preferred methods include embodiments wherein said exosomes are concentrated by affinity chromatography.

Preferred methods include embodiments wherein said exosomes are concentrated by centrifugation.

### DETAILED DESCRIPTION OF THE INVENTION

Within the scope of the invention is generation of “veto cells” using dendritic cell (DC)s as “toxic antigen presenting cells, or in other words, cells that kill T cells or other immune cells that recognize them. Since the DCs are of donor origin, this induces donor specific tolerogenesis.

The terms “polypeptide” and “protein” used herein may be used interchangeably to indicate a string of at least two amino acid residues that are interconnected with one another via covalent bond (e.g., peptide bond), which may be recombinant polypeptides, natural polypeptides, or synthetic polypeptides. Particularly, the polypeptides described herein are human and/or mouse polypeptides.

The terms “variant”, “polypeptide variant” or “analogue” used herein indicates a polypeptide that is different from the original polypeptide in the amino acid sequence by one or more substitutions, deletions, insertions, fusions, truncations or any combinations thereof. The variant polypeptide may be fully functional or may lack one or more active functions. The term “functional variant” used herein only contains, for example, conservative changes or the changes in non-critical residues or non-critical regions, and retains the functions of the original polypeptide. The functional variant may further contain the substitution of similar amino acids, which results in unchanged functions or insignificant function changes. Amino acids that are important for the functions may be identified by methods known in the art, for example, site directed mutagenesis or glycine scanning mutagenesis (Cunningham, B. and Wells, J., Science, 244: 1081-1085, 1989). Sites that are crucial to polypeptide activity may be determined by, for example, structural analysis such as crystallization, nuclear magnetic resonance, or photoaffinity labeling (Smith, L. et al., J. Mol. Biol., 224:899-904, 1992; de Vos, A. et al., Science, 255: 306-312, 1992).

The term “fragment” used herein refers to a molecule that is only a part of a full-length sequence. For example, a Jhdm1b polypeptide fragment is truncated Jhdm1b. The fragments may contain a sequence from any end of the full-length sequence or a sequence from the middle of the full-length sequence. The fragment may be a “functional fragment”, for example, a fragment that retains one or more functions of the full-length polypeptide. The term “functional fragment” used herein indicates that said fragment retains the functions of the full-length polypeptide, for example, inducing pluripotent stem cells or increasing the efficiency of inducing pluripotent stem cells.

Unless otherwise stated, when polypeptides, nucleic acids, or other molecules are mentioned herein, they include functional variants and functional fragments. For example, Jhdm1b and Jhdm1a further indicate the functional variants and functional fragments of natural Jhdm1b and Jhdm1a respectively.

The term “Jhdm1b” used herein may indicate a member of the family of JmjC-domain-containing histone demethylase (JHDM) that is evolutionarily conserved and widely expressed. It is also called Fbx110. In particular, said polypeptide is a human and/or mouse polypeptide.

The term “Jhdm1a” used herein may indicate another member of the family of JmjC-domain-containing histone demethylase (JHDM). It is also called Fbx111. In particular, said polypeptide is a human and/or mouse polypeptide.

The term “induced pluripotent stem cells” or “iPSs” used herein may be used interchangeably to indicate pluripotent stem cells obtained by artificially inducing non-pluripotent cells (such as somatic cells). Said inducing is generally achieved by forced expression of a specific gene, and this process is also called “inducing cells into pluripotent stem cells” herein.

The term “stem cell inducing factor” used herein indicates a factor that is capable of inducing cells into pluripotent stem cells by itself alone or in combination with other factors, such as proteins, polypeptides, and encoded or noncoding RNAs. Preferably, the stem cell inducing factor is a transcription factor, including Oct-3/4, the members of Sox family, the members of Klf family, the members of Myc family, Nanog, LIN28 and the like. Preferably, the stem cell inducing factor is selected from one or more of Oct4, Klf4, Sox2, and c-myc. More preferably, the stem cell inducing factor includes at least Oct4. In particular, the polypeptide is a human and/or mouse polypeptide.

The term “Oct4” used herein indicates a member of the family of octamer transcription factors. It plays a crucial role in maintaining the pluripotency of the cells. In the literatures, Oct4 was also called Oct3.

The term “Klf4” used herein indicates a member of the KrOppel-like family of transcription factors.

The term “Sox2” used herein indicates a member of the family of Sox transcription factors.

The term “c-myc” used herein indicates a transcription factor that is well known by those skilled in the art. It regulates the expression of many genes and recruits histone transacetylase. Its mutations are related to many cancers.

The term “histone modification” used herein indicates a variety of modifications to histone, such as acetylation, methylation, demethylation, phosphorylation, adenylation, ubiquitination, and ADP ribosylation. In particular, the histone modification includes the demethylation of histone.

The term “object” used herein refers to mammals, such as human being. Other animals may also be included, for example domestic animals (e.g., dog and cat), poultry (such as cattle, sheep, swine, and horse), or laboratory animals (such as monkey, rat, mouse, rabbit, and guinea pig).

The term “consistency”, “percent consistency”, “homology”, or “identity” used herein refers to the sequence identity between two amino acid sequences or nucleic acid sequences. The percent consistency may be determined by the alignment of two sequences, and it refers to the number of identical residues (i.e., amino acids or nucleotides) at positions common to the compared sequences. Sequence alignment and comparison may be carried out by the standard algorithms of the art (for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or a computerized version of these algorithms (Wisconsin Genetics Software Package Release 7.0, Genetics Computer Group, 575 Science Drive, Madison, Wis.). The computerized version is publicly available as BLAST and FASTA. Additionally, the ENTREZ available from the National Institute of Health (Bethesda Md.) may be used for sequence comparison. When BLAST and gapped BLAST programs are used, the default parameters of the respective programs (such as BLASTN, which is available on the internet site of the National Center for Biotechnology Information) may be used. In one embodiment, GCG with a gap weight of 1 may be used to determine the percent identity between two sequences, such that each amino acid gap is given a weight as if it is a single amino acid mismatch between the two sequences. Alternatively, ALIGN program (version 2.0), which is a part of GCG (Accelrys, San Diego, Calif.) sequence alignment software package, may be used.

The term “vector” used herein is used in the meaning well known by those skilled in the art and may be an expression vector. The vector may include viruses (such as poxvirus, adenovirus, and baculovirus); yeast vectors, bacteriophages, chromosomes, artificial chromosomes, plasmids, cosmids, episome vectors, and mRNA vectors, or may be chemically synthesized directly. Preferably, the virus vector is a retrovirus and/or lentivirus vector. More preferably, the retrovirus is a pMXs vector.

The term “excessive” used herein indicates being significantly higher than the normal level, and particularly indicates that the expression of a polypeptide is statistically significantly higher that in normal cells. Preferably, it is higher by 20%, 50%, 100%, 200%, or even 5, 10, or 100 times.

The term “over-expression” used herein indicates that the expression level is significantly higher than the normal level, and particularly indicates that the expression of a polypeptide is statistically significantly higher that in normal cells. Preferably, it is higher by 20%, 50%, 100%, 200%, or even 5, 10, or 100 times.

It is known that DCs have different stages in their development during which they, for example, predominately take up antigen, rather than present it. For example, it is thought that DCs may have immature stages characterised by the uptake of large amounts of potential antigens and more mature stages characterised by lower amounts of antigen uptake, but increased amounts of antigen presentation of the antigens they acquired previously. The DCs of the invention are initially immature but may be induced to mature as evidenced by their ability to cross present antigen to naïve or memory CD8+ T cells. Methods for making immature DCs are disclosed in more detail below. The DCs may of course be manipulated in vitro and this may allow control of whether the cells are exposed to stimuli which promote dendritic cell maturation. Thus, by ensuring that the cells are exposed to stimuli responsible for inducing maturation, the resultant cells may be used to promote an immune response when they are transferred to a patient. The DCs of the invention can be identified as dendritic cells using standard methods known in the art, including expression of lineage restricted markers, structural and functional characteristics. The DCs will express detectable levels of cell surface markers known to be characteristic of dendritic cells. In particular the DCs may express detectable levels of any one of CD11c, CD209 (also known as DC sign), CD13, low levels of CD200R, CD11b, CD83 and CD40. The cells may be CD14lo. In some cases the cells may express all of CD11c, CD209 and CD13 and may also be CD14lo. The DCs of the invention typically express MHC molecules. The DCs of the invention typically express both MHC class I and MHC class II. The DCs of the invention preferably express detectable levels of MHC class II. Any of the HLA haplotypes may be present on the DCs of the invention.

Morphologically, DCs are typically characterized by extensive veils of cytoplasm and individual cells with many dendrites. Another defining characteristic of DCs is their ability to stimulate an allogeneic T cell response in a mixed leukocyte reaction (MLR). In an MLR, DCs are cultured together with allogeneic lymphocytes. Due to the histocompatibility mismatch between the cells, T cells recognize allogeneic MHC molecules expressed by DC as foreign and respond by proliferating vigorously in culture. The DCs of the invention are distinguished from known DCs, such as moDCs, in various ways. The DCs of the invention preferably express detectable levels of tolerogenic molecules such as Fas ligand. This cell surface antigen is typically expressed by cells capable of antigen cross presentation.

The DCs of invention preferably express low levels of TLR2 and TLR4 when determined by flow cytometry. In any case, the DCs of the invention preferably respond to ligands of TLR2 and TLR4 in functional assays, such as the secretion of IL-6, detected by ELISA assays. TLR-2 (also known as CD282) is a surface membrane receptor protein which plays a fundamental role in pathogen recognition and activation of innate immunity. This protein is expressed most abundantly in peripheral blood leukocytes, and mediates host response to Gram-positive bacteria and yeast via stimulation of NF-κB. TLR2 is involved in the recognition of a wide range of PAMPs derived from bacteria, fungi, parasites and viruses. These include lipopeptides from bacteria, peptidoglycan and lipoteichoic acid from Gram-positive bacteria, lipoarabinomannan from mycobacteria, zymosan from fungi, tGPI-mucin from Trypanosoma cruzi and the hemagglutinin protein from measles virus. TLR2 generally forms heterodimers with TLR1 or TLR6. Specifically, the TLR2-TLR1 heterodimer recognizes triacylated lipopeptides from Gram-negative bacteria and mycoplasma, whereas the TLR2-TLR6 heterodimer recognizes diacylated lipopeptides from Gram positive bacteria and mycoplasma. The DCs of the invention may be used to treat chronic infections by bacteria, fungi, parasites and viruses. The therapeutic methods of the invention may involve combined therapy with DCs of the invention and one or more lipopeptide, peptidoglycan, lipoteichoic acid, lipoarabinomannan, zymosan, tGPI-mucin and hemagglutinin protein. TLR4 (also known as CD284; Akira, et al., Cell 124, 783-801 (2006)) is a cell surface protein that detects lipopolysaccharide on Gram-negative bacteria and is thus important in the activation of the innate immune system. The DCs of the invention may be used to treat Gram-negative bacterial infections. The therapeutic methods of the invention may involve combined therapy with DCs of the invention and one or more bacterial lipopolysaccharides. Standard methods known in the art may be used to determine the detectable expression, low expression or lack thereof of the various markers discussed above. Suitable methods include, but are not limited to, immunocytochemistry, flow cytometry and quantitative PCR. DC responses to TLR ligands may also be measured using standard assays known in the art. Suitable methods include, but are not limited to, enzyme-linked immunosorbent assays (ELISA) for the secretion of cytokines, enhanced mixed leukocyte reactions and up-regulation of co-stimulatory molecules and maturation markers, measured by flow cytometry.

The ability of the DCs of the invention to cross-present antigen may be tested using any assay known in the art. The DCs of the invention may be loaded or transfected with the antigen as discussed in more detail below. The DCs of the invention are typically loaded with or cultured in the presence of a test antigen and the ability of the cells to present a peptide derived from the test antigen using MHC class I molecules is determined Presentation may be measured by culturing the cross-presenting DCs with appropriate CD8+ HLA-restricted T lymphocytes which have been primed with the antigen in question and determining whether or not such T lymphocytes produce IFN-γ. Alternatively, well-characterised MHC class I-restricted T cell clones of known antigen specificity may be used as a readout for cross-presentation of whole exogenous antigen. A DC of the invention may be isolated, substantially isolated, purified or substantially purified. The DC is isolated or purified if it is completely free of any other components, such as culture medium, other cells of the invention or other cell types. The DC is substantially isolated if it is mixed with carriers or diluents, such as culture medium, which will not interfere with its intended use. Alternatively, the DC of the invention may be present in a growth matrix or immobilized on a surface as discussed below. DCs of the invention may be isolated using a variety of techniques including antibody-based techniques. Cells may be isolated using negative and positive selection techniques based on the binding of monoclonal antibodies to those surface markers which are present on the DC. Hence, the DCs may be separated using any antibody-based technique, including FACS and magnetic bead separation.

In one embodiment the present invention provides transformed immune cells generated in a donor specific manner using pluripotent stem cell generating technologies, such as inducible pluripotent stem cell technology. These technologies are combined with transfection of staid DC or DC-like cell with toleogenic molecules. Furthermore, iPSC and/or derivatives of said iPSC differentiated into the DC lineag may be engineered in a manner that exhibit a gene specific targeted knock-out phenotype. Such transformed immune cells can be used in a variety of therapeutic in vitro, ex vivo and in vivo methods to modulate T cell activity and thus have use in therapeutic approaches for the treatment of immune disorders in mammalian subjects. he immune cells of the invention exhibit a targeted gene-specific knockout phenotype which may be accomplished using any technique that provides for the targeted silencing of an endogenous gene. In one aspect of the invention the technique of RNAi (RNA interference) was used to create transformed immune cells suitable for use for the modulation of T cell activity in vitro, ex vivo or in vivo. In this aspect, the immune cells are transfected with a siRNA (small interfering RNA) designed to target and thus to degrade a desired mRNA in order not to express the encoded protein that is involved in T cell activity. Thus such transfected immune cells may be used to suppress or stimulate immune system functioning via the modulation of T cell activity. It is understood by those of skill in the art that any method for silencing a specific gene may be used in the present invention. Representative examples of suitable techniques include but are not limited to RNAi and hybrid DNA/RNA constructs. The hybrid DNA/RNA constructs are essentially siRNA constructs in which the nucleic acid composition used for silencing is altered to include DNA

Immune cells for use in the present invention may be selected from antigen presenting cells (APC) and endothelial cells. Both APC and endothelial cells are known to be able to activate T cells, or in the case preferred by the inventors in the present invention, to induce T cell death, T cell anergy, or Treg differentiation. . . . In preferred embodiments of the invention, the immune cells are APC that may be selected from the group consisting of macrophages, myeloid cells, B lymphocytes, DC and mixtures thereof. It is also within the scope of the present invention to use other APC capable of inducing T cell inhibition through the T cell receptor as is understood by one of skill in the art. In particularly preferred embodiments of the invention, the immune cell is a DC. APC such as DC are known to be phagocytic in nature and thus tend to take up molecules within their environment. In the present invention DC is specifically demonstrated to be successfully altered with siRNA to exhibit a stable phenotype. Therefore one of skill in the art would readily understand that any APC may be altered in accordance with the present invention and used in the methods of the invention. It is also understood that a combination of different types of immune cells may be used in the methods of the present invention. According to an embodiment of the invention, DC are transformed with a designed siRNA. In this embodiment DC must be isolated from a subject and expanded in vitro. DC are typically derived from a source such as bone marrow, peripheral blood, spleen and lymph. In a preferred embodiment DC are generated from iPSC cells. Generation of DCs from iPSC is known in the art and various means disclosed previously may be utilized. In one embodiment, the means described below are utilized. Induced pluripotent stem cells and methods of producing them are utilized as known in the art. A method for inducing pluripotency of differentiated cells, such as somatic cells, was first disclosed by Yamanaka (WO 2007/069666). In this method, somatic cells are reprogrammed using three main nuclear reprogramming factors, namely an Oct family gene, a K1f family gene and a Sox family gene (preferably Sox2). The factors are preferably Oct3/4, K1f4 and Sox2. A fourth reprogramming factor, namely the product of a Myc family gene (preferably c-Myc), may also be used. Numerous different methods have since been disclosed for inducing pluripotency in somatic cells. In some embodiments, various are performed such as culture of the “mother cells” in the presence of a histone deacetylase inhibitor. Suitable histone deacetylase inhibitors include valproic acid, sodium phenylbutyrate, trichostatin A, sulforaphane, and phenylbutyrate.

For the purpose of the invention, the generated iPSCs cells typically display the characteristic morphology of human embryonic stem cells (hESCs), express the pluripotency-associated markers SSEA-4 and TRA1-60, the transcription factors Oct-4 and Nanog and differentiate in vitro into cell types derived from each of the three embryonic germ layers. The iPSCs may be an established cell line. More preferably, the iPSCs are produced from somatic cells taken from a patient to be treated in accordance with the invention. The iPSCs may be derived from any human somatic cell. Suitable cells include, but are not limited to, keratinocytes, dermal fibroblasts or leukocytes derived from peripheral blood. The iPSCs are preferably derived from dermal fibroblasts. Techniques for culturing iPSCs are well known to a person skilled in the art. Suitable conditions are discussed above. Conditions suitable for inducing stem cells to differentiate into DCs are known in the art. For instance, suitable conditions are disclosed in Tseng, S-Y. et al. Regen. Med. 4, 513-526 (2009). However, it is surprising that culturing human iPSCs under these condition results in DCs that are capable of cross presenting an antigen to naïve CD8+ T lymphocytes.

In a preferred embodiment, the method comprises (a) culturing the iPSCs in a medium comprising granulocyte macrophage-colony stimulating factor (GM-CSF) for sufficient time to produce monocytic cells, (b) culturing the monocytic cells under conditions that induce the formation of immature dendritic cells and (c) culturing the immature dendritic cells in a medium comprising growth factors that induce maturation of the dendritic cells. The sufficient time in step (a) is typically from 13 to 17 days. In step (a), the medium preferably further comprises one or more of stem cell factor (SCF), vascular endothelial growth factor (VEGF) and bone morphogenic protein (BMP-4). The medium more preferably initially comprises all three of SCF, VEGF and BMP-4 and each is successively removed. Step (a) most preferably comprises initially culturing the iPSCs in a medium comprising GM-CSF, SCF, VEGF and BMP-4, removing BMP-4 from day 5 onwards, removing VEGF from day 14 onwards and removing SCF from day 19 onwards until monocytic cells are produced. The sufficient time in step (b) is typically from 9 to 15 days. Suitable conditions for forming immature DCs from monocytic cells are known in the art. Step (b) preferably involves culturing the monocytic cells in a medium comprising GM-CSF and interleukin-4 (IL-4) for sufficient time to produce immature DCs. Step (c) takes from 36 hours to 4 days, preferably about 2 days (48 hours). The medium in step (c) preferably comprises GM-CSF, tumor necrosis factor-α (TNFα), prostaglandin-E2 (PGE2), interleukin-1β (IL-1β) and interferon-γ (IFNγ). Steps (a) to (c) typically take from 21 to 32 days. Preferred concentrations of the various growth factors are as follows: GM-CSF—from 25 to 75 ng/ml, more preferably 50 ng/ml; SCF—from 10 to 30 ng/ml, more preferably 20 ng/ml; VEGF—from 25 to 75 ng/ml, more preferably 50 ng/ml; BMP-4—from 25 to 75 ng/ml, more preferably 50 ng/ml; IL-4—from 10 to 150 ng/ml, more preferably 25 or 100 ng/ml; TNFα—from 10 to 30 ng/ml, more preferably 20 ng/ml; PGE2—from 0.5 to 1.5 ng/ml, more preferably 1.0 ng/ml; IL-1β—from 5 to 15 ng/ml, more preferably 10 ng/ml; and IFNγ—from 10 to 20 ng/ml, more preferably 15 ng/ml. The growth factors used in the method of the invention are typically the human forms. The growth factors used in the method of the invention are typically recombinant. The use of such factors means that the DCs of the invention are produced in clinically relevant conditions, i.e. in the absence of trace amounts of endotoxins and other environmental contaminants, such as lipopolysaccharides, lipopeptides and peptidoglycans, etc. This makes the DCs of the invention particularly suitable for administration to patients. The method preferably further comprises isolating the DCs of the invention. Any of the methods discussed above may be used. The invention also provides a method for producing a population of the invention that is suitable for administration to a patient, wherein the method comprises producing iPSCs from somatic cells obtained from the patient and producing a population of the invention from those iPSCs using the method of the invention described above. The population will be autologous with the patient and therefore will not be rejected upon implantation. The invention also provides a population of the invention that is suitable for administration to a patient and is produced in this manner. Alternatively, the invention provides a method for producing a population of the invention that is suitable for administration to a patient, wherein the method comprises the differentiation of partially-matched iPSCs obtained from a public bank of clinically-approved lines. Substances which stimulate hematopoiesis (i.e. G-CSF and GM-CSF) may be first administered to the subject in order to increase the number of DC. Blood is treated to isolate the DC from other cell types by standard methods known in the art. Isolated DC cultured in vitro may be treated with cytokines to increase their number. Methods for isolating and ex vivo culture of DC are known in the art and described for example in U.S. Pat. Nos. 5,199,942, 5,851,756, 6,017,527, 6,251,665, 6,458,585 and 6,475,483 (the disclosures of which are incorporated herein by reference in their entirety). The present invention also encompasses therapeutic methods for the treatment of a variety of immune disorders in a mammalian subject. The methods may involve the use of a siRNA designed for use directly in vivo to block the expression of a gene by an immune cell, the gene expressing a protein involved in the activity of T cells which elicits an immune disorder. Alternatively, the methods may involve the use of an immune cell which contains at least one double-stranded RNA molecule (siRNA) that inhibits the expression of an endogenous target gene encoding a surface marker, a chemokine, a cytokine, an enzyme or a transcriptional factor. In preferred embodiments of the invention, the methods of the invention comprise the use of an altered (i.e. transformed) DC that contains a double-stranded RNA molecule that inhibits the expression of an endogenous target gene encoding a surface marker, a chemokine, a cytokine, an enzyme or a transcriptional factor. Still in other embodiments, the therapeutic method may involve ex vivo treatment of tissues and/or organs intended for transplantation. In aspects of the invention, the siRNA possesses specific homology to part or to the entire exon region of a surface marker, a chemokine, a cytokine, an enzyme or a transcriptional factor normally expressed by the immune cell such that the gene is silenced It is understood by one of skill in the art that the siRNA as herein described may also include altered siRNA that is a hybrid DNA/RNA construct or any equivalent thereof. In preferred embodiments of the invention the transfected DC cells are prepared by the method of RNAi. RNA interference is a mechanism of post-transcriptional gene silencing. Specific gene silencing is mediated by short strands of duplex RNA of approximately 21 nucleotides in length (termed small interfering RNA or siRNA) that target the cognate mRNA sequence for degradation. While many techniques have been used to block specific molecules in vitro and in vivo, such as anti-sense oligonucleotides (Gerwitz, A. M. 1999. Curr Opin Mol Ther 1:297) and monoclonal antibodies (Drewe, E., et al., 2002. J Clin Pathol 55:81), RNAi was used in the present invention because it provides several distinct advantages. First, mRNA degradation by siRNA is extremely efficient as only a few copies of dsRNA are necessary to activate the RNA Induced silencing complex (RISC) (Martinez, J. A. et al., 2002. Cell 10:563). Once RISC is activated it can conduct multiple rounds of gene-specific mRNA cleavage. Second, RNAi is specific, in that only sequences with identity to one of the strands of dsRNA will be cleaved (Hannon, G. J. 2002. Nature 418:244). Third, the RNAi effect is long lasting and can be spread to progeny cells after replication, although a dilution effect is evident in mammalian cells (Fire, A., et al., 1998. Nature 391:806). This technique is relatively simple, giving rise to an in vitro knock down phenotype within days that can be confirmed with many antibody based detection systems (such as ELISA or Western Blotting), or if an antibody is not available, by RT-PCR or functional assays. DC may be transformed with siRNA alone, siRNA contained within a plasmid or vector that results in the production of the siRNA, siRNA contained within a plasmid or vector that further expresses a selected antigen and siRNA together with a mRNA from a tumor cell. In the case of the plasmid or is vector further expressing a selected antigen, the DC will process or modify the antigen in a manner-to promote the stimulation of T cell activity by the processed or modified antigens. Methods for making siRNA and cell transformation are described for example in U.S. Patent Application 2002/0173478, U.S. Patent Application 2002/0162126, PCT/US01/10188, PCT/EP01/13968 and in Simeoni F., et al., 2003 Nucleic Acids Res June 1; 31(11):2717-24 (the disclosures of which are incorporated herein in their entirety). Methods for producing antigen pulsed DC are known and exemplified for example in U.S. Pat. Nos. 6,497,876 and 6,479,286 (the disclosures of which are incorporated herein by reference in their entirety). Methods for making siRNA plasmids or vectors are also known and described for example in U.S. Patent Application 2003/0104401, in Morris M. C., et al., 1997. Nucleic Acid Res. July 15:25(14):2730-6 and in Van De Wetering M., et al., 2003, EMBO June; 4(6):609-15 (the disclosures of which are incorporated herein in their entirety). Suitable lipid-based vectors may include but are not limited to lipofectamine, lipofectin, oligofectamine and GenePorter™. Methods for producing tumor derived RNA for pulsing DC are also known to those of skill in the art and are described for example in U.S. Patent Application 2002/0018769 (the disclosure of which is incorporated herein in its entirety). In embodiments of the invention, DC are transformed to contain a double-stranded RNA molecule that inhibits the expression of an endogenous target gene encoding a protein that either suppresses T cell activation or alternatively stimulates T cell activation. For the suppression of T cell activation, the immune cells of the invention are transformed with a double-stranded RNA molecule that inhibits the expression of a gene that encodes a co-stimulatory molecule, cytokine, adhesion molecule, enzyme or transcription factor. Representative examples of such co-stimulatory molecules, cytokines, adhesion molecules, enzymes and transcription factors may be selected from the group consisting of TNFα, IL-1, IL-1b, IL-2, TNFβ, IL-6, IL-7, IL-8, IL-23, IL-15, IL18, IL-12, IFNγ, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR-4, TLR-9, 4-1 BBL, nicotinic receptor, GITR-L, OX-40L, CD-CK1, TARC/CCL17, CCL3, CCL4, CXCL9, CXCL10, IKK-β, NF-κB, STAT4, ICSBP/IFN, regulatory factor 8, TRAIL, Inos, arginase, FcgammaRI and II, thrombin, MIP-1α and MIP-1B.

1. A method of inducing antigen specific tolerance to a transplanted organ or collection of cells comprising:
a) identifying a patient in need of an organ or cellular transpant and a donor of said organ or collection of cells;
b) creating and extracting pluripotent stem cells from the donor;
c) inducing differentiation of said extracted pluripotent stem cells into cells resembling cells possessing tolerogenic properties; and
d) administering said cells into a patient in need of an organ or cellular transplant.

2. The method of claim 1, wherein the pluripotent stem cells are induced by contacting said cells with interleukin-4 and GM-CSF.

3. The method of claim 1, wherein said dendritic cells are resistant to maturation stimuli.

4. The method of claim 3, wherein resistance to maturation is achieved by gene editing to remove NF-kappa B.

5. The method of claim 3, wherein resistance to maturation is achieved by gene editing to constitutively express i-kappa B.

6. The method of claim 3, wherein resistance to maturation is achieved by gene editing to remove rel-B.

7. The method of claim 3, wherein resistance to maturation is achieved by gene editing to remove CD40.

8. The method of claim 3, wherein resistance to maturation is achieved by gene editing to remove CD80.

9. The method of claim 3, wherein resistance to maturation is achieved by gene editing to remove CD86.

10. The method of claim 3, wherein resistance to maturation is achieved by gene editing to remove interleukin-7.

11. The method of claim 3, wherein resistance to maturation is achieved by gene editing to remove interleukin-12.

12. The method of claim 3, wherein resistance to maturation is achieved by gene editing to remove interleukin-15.

13. The method of claim 3, wherein resistance to maturation is achieved by gene editing to remove interleukin-18.

14. The method of claim 3, wherein resistance to maturation is achieved by gene editing to remove interleukin-21.

15. The method of claim 3, wherein resistance to maturation is achieved by gene editing to remove interleukin-23.

16. The method of claim 3, wherein resistance to maturation is achieved by gene editing to remove interleukin-27.

17. The method of claim 3, wherein resistance to maturation is achieved by gene editing to remove interleukin-17.

18. The method of claim 1, wherein said dendritic cells are capable of cross presentation to CD8 T cells.

19. The method of claim 18, wherein said CD8 T cells are capable of producing granzyme B upon CD3 crosslinking.

20. The method of claim 18, wherein said CD8 T cells are capable of producing complement C3 upon CD3 stimulation.
